(19) United States
(12) Patent Application Publication
(10) Pub. No.: US 2010/0152107 A1
(43) Pub. Date: Jun. 17, 2010

(54) LONG-TERM FEED - CANCER PATIENT

(75) Inventors: Herve Le-Henand, Saint Germain en Laye (FR); Francois Murbach, Divonne les Bains (FR); Michael Jedwab, Lausanne (CH); Claudia Roessle, Morges (CH); Luc Adam Cynober, Scasux (FR); Stepher Michel Scnneider, Nice (FR)

Correspondence Address: Nestle HealthCare Nutrition, 12 Vreeland Road, 2nd Floor, Box 697, Florham Park, NJ, 07932, US

(73) Assignee: NESTEC S.A., Vevey (CH)

(21) Appl. No.: 12/444,938

(22) PCT Filed: Oct. 17, 2007
(86) PCT No.: PCT/EP2007/061108

§ 371 Date: Mar. 1, 2010

Related U.S. Application Data
(60) Provisional application No. 60/862,159, filed on Oct. 19, 2006.

(51) Int. Cl. A61K 38/02; A61P 3/02; A61P 3/00

(52) U.S. Cl. 514/12

(57) ABSTRACT

Pursuant to the present invention, methods and compositions are provided for providing long-term tube-fed nutrition. More specifically, pursuant to the present invention, methods and compositions are provided for providing long-term tube-fed nutrition to cancer patients.

### BACKGROUND

The present application relates to nutrition. More specifically, the present invention relates to clinical nutrition.

Due to a variety of diseases, insults, and complications, patients may not be able to obtain the necessary nutrition by ingesting food through the mouth, e.g., eating food. Therefore, it has been known to provide clinical nutrition either enterally or parenterally. A variety of different formulations have been developed to provide such clinical nutrition.

Even with respect to typical enteral nutritional products, these products are designed for short-term use, typically 10 to 24 days. In this regard, the products usually provide the essential nutritional components to provide necessary nutrition to patients having acute pathologies during their hospital stays. Although these products are suitable for such short term use, they have not necessarily been designed for long-term feeding of patients. With advances in medicine resulting in increased life expectancy and better disease treatments, a number of individuals could benefit from products designed to provide long-term enteral nutrition.

### SUMMARY

Pursuant to the present invention, methods and compositions are provided for supplying long-term tube-fed nutrition. More specifically, pursuant to the present invention, methods and compositions are provided for providing long-term tube-fed nutrition to cancer patients.

To this end, in an embodiment, a method for providing long term nutrition to a cancer patient comprises the steps of administering at least once a day long term through a tube to a cancer patient a nutritional product comprising: a source of protein which provides 14 to 25% by caloric content of the product wherein the protein source comprises at least 50% by caloric content whey; at least 0.5% by caloric content glutamine; at least 0.8% by caloric content leucine; a source of carbohydrates that comprises 20 to 55% by caloric content of the product; and a source of lipids that comprises 25 to 40% by caloric content of the product.

In an embodiment, glutamine comprises 0.5 to 10.0% by caloric content of the product. The glutamine or a part of it can be packaged in a separate container from remaining components of the product. The glutamine can be free as an amino acid or provided as a glutamine-rich compound such as a dipeptide. The glutamine can comprise alanyl-glutamine.

Pursuant to the method the protein source can be intact or partially hydrolyzed protein. The protein source can be 100% whey protein.

The lipid source can provide 30 to 35% by caloric content of the product. The energy density of the product is between 1.0 to 2.0 kcal. Additionally, the product can comprise a source of fiber.

In an embodiment, 1.5 to 3.5% by caloric content of the product is provided by leucine.

In an embodiment of the method, the product comprises sufficient vitamins and minerals to meet at least one government regulation selected from the group consisting of: U.S. RDA, French RDA, and German RDA.

Pursuant to an embodiment of the method, the patient receives a second different tube-fed nutritional product once normo-metabolic status has been regained. In another embodiment, the patient receives the second different tube-fed product before cancer is diagnosed.

In another embodiment of the present invention, a method of providing long term tube-fed nutrition to a cancer patient is provided comprising the steps of: administering through a tube to a cancer patient during an inflammatory phase and a weight gain phase a first composition comprising: a source of protein which provides 14 to 25% by caloric content of the product wherein the protein source comprises at least 50% by caloric content whey, at least 0.5% by caloric content glutamine, at least 0.8% by caloric content leucine, a source of carbohydrates that comprises 20 to 55% by caloric content of the product, and a source of lipids that comprises 25 to 40% by caloric content of the product; and administering through a tube to a cancer patient during a normo-metabolic phase a second long-term nutritional composition that has a different composition than the first composition.

In an embodiment of the method, the second long-term tube-fed nutrition composition comprises per 100 kcal of product: a source of protein; a source of carbohydrates; a source of lipids; sodium 100 to 200 mg; potassium 25 to 250 mg; calcium above 50 mg; phosphorus less than 150 mg; magnesium at least 15 mg; chloride at least 100 mg; iron 0.4 to 1.5 mg; zinc 0.4 to 2.0 mg; copper 0.08 to 0.4 mg; fluoride 0 to 0.15 mg; chromium 2.0 to 10.0 micrograms; molybdenum 2.0 to 14.0 micrograms; selenium 3.0 to 9.0 micrograms; manganese 0.1 to 0.4 mg; iodine 7.0 to 15.0 micrograms; Vit A 100 to 500 IU; Vit D 0.5 to 2.5 micrograms; Vit E 1.5 to 4.0 mg; Vit K greater than 4.0 micrograms; Vit C greater than 4.0 mg; Vit B1 greater than 0.06 mg; Vit B2 greater than 0.07 mg; Vit B3 0.7 to 3.5 mg; Vit B5 0.2 to 2.0 mg; Vit B6 0.1 to 0.7 mg; Vit B8 at least 1.0 micrograms; Vit B9 at least 12.0 micrograms; and Vit B12 0.1 to 1.0 micrograms.

In an embodiment of the method, the glutamine comprises 0.5 to 10.0% by caloric content of the product. The glutamine or part of it can be packaged in a separate container from remaining components of the product.

In yet a further embodiment of the present invention, a method of treating a cancer patient is provided comprising the steps of: administering at least once a day long term a first nutritional composition comprising a source of protein which provides 14 to 25% by caloric content of the product wherein the protein source comprises at least 50% by caloric content whey; a source of carbohydrates that comprises 20 to 55% by caloric content of the product; and a source of lipids that comprises 25 to 40% by caloric content of the product; and prior to a cancer treatment selected from the group consisting of chemotherapy and radiation therapy administering along with the first nutritional composition glutamine at a level of at least 0.5% of the total caloric intake.

In an embodiment, the method can include the step of administering to the patient after the treatment the first nutrition composition without co-administration of glutamine.

Still further, in an embodiment of the present invention, a composition for providing long term nutrition through a tube to a cancer patient is provided comprising: a source of protein which provides 14 to 25% by caloric content of the product wherein the protein source comprises at least 50% by caloric content whey; at least 0.5% by caloric content glutamine; at least 0.8% by caloric content leucine; a source of carbohydrates that comprises 20 to 55% by caloric content of the product; and a source of lipids that comprises 25 to 40% by caloric content of the product.

An advantage of the present invention is to provide improved enteral nutrition products.

Another advantage of the present invention is to provide improved methods of providing enteral nutrition.

Furthermore, an advantage of the present invention is to provide enteral nutrition compositions for providing long-term tube-fed nutrition to a cancer patient.

Moreover, an advantage of the present invention is to provide methods of providing long-term tube-fed enteral nutrition to cancer patients.

Additional features and advantages are described herein, and will be apparent from, the following Detailed Description.

### DETAILED DESCRIPTION

The present invention relates to clinical nutrition. More specifically, the present invention relates to providing long-term tube-fed nutrition to patients. As used herein, the term “long-term” means greater than one month (30 days). As used herein, the term “tube-fed” means to provide a product to a patient through a feed tube that is received within a portion of the digestive tract of a patient, for example, a nasogastric feed tube or a percutaneous endoscopic gastrostomy tube. Applicants are filing herewith a patent application entitled “METHODS OF PROVIDING LONG-TERM NUTRITION,” the disclosure of which is incorporated herein by reference.

The long-term tube-fed nutrition products are preferably designed for cancer patients. As used herein, “cancer patient” refers to a patient who cannot receive nutrition through a normal diet or is malnourished and who is suffering from an active cancer, i.e., is not normo-metabolic. The active cancer may be a cancer of the neck, head, or digestive tract, or it may be a recurrence of cancer in a patient who has previously suffered and been treated for a cancer of the head, neck or digestive tract which has left him or her unable to eat a normal diet. As used herein, the term “normal diet” means to receive at least substantially all nutrition by eating, i.e., using one's mouth, without the use of any feed tube or parenteral feed.

The present invention provides a nutritional formulation that, in part, will benefit patients long term as they suffer from cancer and especially during their cancer treatment, e.g., chemotherapy or radiation therapy. The formulation provides benefits also during the inflammatory and/or hypercatabolic episodes as compared to standard enteral nutrition products. As used herein, the term “standard enteral nutrition product” refers to products that are not specifically advertised or promoted for long-term use. A variety of such products are available, for example, from Nesté, Abbott, Novartis, Numico, and Fresenius. Therefore, the product, in part, is hyperprotinic and hypercaloric.

In part, the formulation is enriched in, preferably, w3 polyunsaturated fatty acids (EPA and DHA). These fatty acids provide good anti-inflammatory characteristics, especially for patients suffering from tumors. Preferably, the protein source comes from whey, specifically proteins rich in cysteine having antioxidant properties and therefore being anti-inflammatory. In part, the anti-oxidant properties are provided by the glutathione synthesis which requires cysteine and glutamine as precursors.

The formula is designed to be utilized, at least at times, with added glutamine. Glutamine can be part of the formulation or it can be provided as a separate component. In this regard, the glutamine can be provided as a module. The module can contain a powder or liquid form of glutamine. With respect to the glutamine and other modules, reference is made to co-pending U.S. patent application entitled: “NUTRITIONAL MODULES,” filed on Oct. 7, 2002, the disclosure of which is incorporated herein by reference. By way of example, a 30 g module of glutamine can be used with the 1500 ml of formula.

Pursuant to an embodiment of the method of the present invention, added glutamine is administered to the patient before the start of a cycle of chemo- or radio-therapy treatment, for example, a week before, and administered throughout the cycle and for a period thereafter, for example, two weeks after treatment has stopped. During the administration of the glutamine, the formulation will also be administered either with the glutamine or separate therefrom. In an embodiment, once acute inflammation as evidenced, for example, by mucositis has subsided, the glutamine administration can stop. The module presentation of glutamine may conveniently be used to complement the formulation during cycles of treatment either in hospital or in a non-hospital setting, for example, home healthcare, and nursing home.

In addition, L-leucine is added to the formula in an amount so as to contribute from 0.8 to 5% of the energy content of the formula. L-leucine is a powerful stimulator of synthesis of protein in muscles in synthesis.

Pursuant to an embodiment of the present invention, prior to the diagnosis of a tumour requiring treatment, if the patient requires enteral nutrition, specifically long-term enteral nutrition, preferably a maintenance formulation is utilized as described in U.S. patent application entitled “LONG-TERM ENTERAL NUTRITION-MAINTENANCE,” that is being filed herewith, the disclosure of which is incorporated herein by reference. Likewise, feeding with the maintenance formulation may be resumed once the cancer is in remission and the patient has achieved a normo-metabolic status.

Accordingly, the present invention provides long-term enteral nutrition for cancer patients that includes providing two distinct formulations, one directed to cancer patients with a tumor requiring treatment and a second for maintenance of patients once normo-metabolic status has been re-established. As used herein, “maintenance patient” refers to an adult patient under the age of sixty-five who cannot receive nutrition through a normal diet but who is normo-metabolic (i.e. not suffering from a metabolic disorder). For the sake of clarity, Applicants note that this application discloses the cancer patient formulation except in Examples 3 and 4, as well as certain claims, wherein the maintenance patient formulation is set forth.

In an embodiment, the formulation of the present invention can be used to reduce the side effects of cancer and its treatment. Such side effects include cachexia due to the cancer and mucositis which may be apparent during treatments such as chemotherapy and radiation therapy.

In an embodiment, the composition includes the following features a source of protein providing 14 to 25 percent of the total energy of the product, preferably 14 to 25 percent can be intact or partially hydrolysed protein of which at least 50 percent is whey. In an embodiment, the protein is 100 percent whey and the protein source, including added amino acids, provides 15 to 30 percent of the total energy of the product, preferably 20 to 27%. In a preferred embodiment, 3 to 7 percent of the energy is provided by glutamine and 1.5 to 3.5 percent of the energy is provided by leucine. By providing 100 percent whey protein an easily absorbed and relatively high cysteine content is provided. By supplementing this content with leucine, one can address the increased requirements for same in this population group.

A source of carbohydrates is preferably provided comprising 20 to 55 percent of the total energy of the product. Any carbohydrate or mixture of carbohydrates can be used. Examples include starch, maltodextrins, sucrose, and mixtures thereof. In an embodiment, 100 percent maltodextrin is used.

In addition, the source of lipids provides 25 to 40 percent of the total energy of the product. Preferably, 1.0 to 7 percent by weight of the source of lipids is eicosapentaenoic acid. By providing EPA, a suitable n6:n3 ratio can be provided since these patients are suffering from inflammation and oxidative stress. Any suitable mixture of dietary lipids can be used. These include saturated fatty acids (SFA), monounsaturated fatty acids (MUFA), polyunsaturated fatty acids (PUFA), and medium-chain triglycerides (MCT). Preferably, the lipid source provides 30 to 35 percent of the total caloric content of the product. It should be noted that, preferably, the energy density of the composition is 1.0 to 2.0 kcal/ml.

The present invention provides methods as well as products that are optimized and/or improved for long-term use. In an embodiment, these products are provided to the patient outside of a hospital setting. For example, the products can be provided in a nursing home, daycare or other out-patient facilities, or even the home of the patient. Preferably, the nutrition products are housed in a plastic bag. A variety of such bags are known, for example, 500 ml, 1000 ml, and 1500 ml bags are known in the art. It should be noted, however, that any suitable container can be used to house the nutrition product. In an embodiment, the product is designed to provide necessary nutrition at 1500 ml per day, although those skilled in the art will appreciate that variations to this level are possible.

Preferably, the products include the necessary nutritional components to provide complete nutrition to the patient on a long-term basis. In this regard, the products include, among other possible ingredients: protein, carbohydrate, fat, vitamins, and minerals. In an embodiment, the products substantially, if not completely, comply with at least certain governmental requirements. As used herein, “governmental requirements” means any recommendations from any one of the following governments: U.S., typically the USRDA, German, typically the German RDA, and French, typically the French RDA. In an embodiment, the nutrition product meets or exceeds at least one of the governmental requirements.

By way of example and not limitation, examples of the present invention will now be given.

Example Nos. 1 and 2 are designed for use from diagnosis to the point where the tumor is in remission.

### Example No. 1

| | | Embodiment | |
|---|---|---|---|
| | | Formula | Embodiment |
| | | 1500 ml | per 100 ml |
| Calories | Kcal | 2310 | 154.00 |
| Protein | G | 129 | 8.6 |
| total) (includes | | | |
| free amino | | | |
| acids) | | | |
| Whey protein | G | 94 | 6.27 |
| hydrolysate | | | |
| added Leucine | G | 5 | 0.33 |
| added | G | 30 | 2.0 |
| Glutamine | | | |
| Carbohydrates | G | 273 | 18.2 |
| Maltodextrins | G | 254 | 16.9 |
| Starch | G | 15 | 1.0 |
| Carbohydrates | G | 4 | 0.27 |
| from other | | | |
| sources | | | |
| Fiber | | | |
| Lipids | G | 77 | 5.1 |
| SFA (includes | G | 34 | 2.3 |
| MCT) | | | |
| MUFA | G | 17 | 1.1 |
| PUFA | G | 20 | 1.3 |
| linoleic acid (n- | G | 14 | 0.92 |
| 6) | | | |
| α linolenic acid | G | 2.0 | 0.13 |
| (n-3) | | | |
| Ratio ω6/ω3 | | 2.5 | 2.5 |
| EPA | G | 2.1 | 0.14 |
| DHA | G | 1.5 | 0.10 |
| Minerals and | | | |
| Trace | | | |
| Elements | | | |
| Sodium | Mg | 2295 | 153.0 |
| Potassium | Mg | 2700 | 180.0 |
| Calcium) | Mg | 1140 | 76.0 |
| Phosphorous | Mg | 1275 | 85.0 |
| Magnesium | Mg | 405 | 27.0 |
| Chloride | Mg | 2070 | 138.0 |
| Iron | Mg | 14 | 0.93 |
| Zinc | Mg | 28 | 1.9 |
| Copper | Mg | 2.4 | 0.16 |
| Fluoride | Mg | 1.95 | 0.13 |
| Chromium | μg | 115.5 | 7.7 |
| Molybdenum | μg | 285 | 19 |
| Selenium | μg | 135 | 9 |
| Manganese | Mg | 5.4 | 0.36 |
| Iodine | μg | 300 | 20 |
| Vitamins | | | |
| Vitamin A total | IU | 8250 | 550 |
| Vitamin D | μg | 30 | 2.0 |
| Vitamin E | IU | 97.5 | 6.5 |
| Vitamin K | μg | 180 | 12 |
| Vitamin C | Mg | 435 | 29 |
| Vitamin B1 | Mg | 4.05 | 0.27 |
| (Thiamin) | | | |
| Vitamin B2 | Mg | 4.35 | 0.29 |
| (Riboflavin) | | | |
| Vitamin B3-PP | Mg | 40.5 | 2.7 |
| (Niacin) | | | |
| Vitamin B5 | Mg | 16.5 | 1.1 |
| (Pantothenic | | | |
| acid) | | | |
| Vitamin B6 | Mg | 5.25 | 0.35 |
| (Pyridoxine) | | | |
| Vitamin B8 | μg | 107 | 7.1 |
| (Biotin) | | | |
| Vitamin B9 | μg | 840 | 56 |
| (Folic Acid) | | | |
| Vitamin B12 | μg | 13.8 | 0.92 |

### Example No. 2

| | | Embodiment | RANGE for | Embodiment |
|---|---|---|---|---|
| | | 1500 ml | 100 kcal | per 100 ml |
| Calories) | kcal | 2310 | preferably | 154.00 |
| | | | 1-2 kcal/ml | |
| Protein total | g | 129 | 15-30% of | 8.6 |
| (includes free | | | total energy | |
| amino acids) | | | content, | |
| | | | preferably 20-27% | |
| | | | of total | |
| | | | energy content, | |
| | | | 14-25% of | |
| | | | total energy | |
| | | | content is | |
| | | | intact or | |
| | | | hydrolysed | |
| | | | protein | |
| | | | of which 50% | |
| | | | whey | |
| Whey protein | G | 94 | | 6.27 |
| hydrolysate) | | | | |
| added Leucine | g | 5 | leucine: 0.8-5% | 0.33 |
| | | | (free, | |
| | | | peptide or | |
| | | | bound) | |
| | | | preferably 1.5-3.5% | |
| | | | of TEI | |
| | | | composition | |
| added | g | 30 | 0.5-10% of the | 2.00 |
| Glutamine | | | energy of the | |
| | | | composition | |
| | | | comes from | |
| | | | glutamine | |
| | | | (free, bound or | |
| | | | short peptides), | |
| | | | preferably 3-7% | |
| Carbohydrate | g | 273 | 20-55% of | 18.2 |
| | | | total energy | |
| | | | content | |
| Maltodextrins | g | 254 | | 16.9 |
| Starch | | 15 | | 1.0 |
| Carbohydrates | | 4 | | 0.27 |
| from other | | | | |
| sources | | | | |
| Fiber | g | | Optional, but if | |
| | | | present, | |
| | | | >10 g/litre | |
| Lipids | | 77 | 25-40% of | 5.1 |
| | | | total energy | |
| | | | content, | |
| | | | preferably 30-35% | |
| SFA (includes | g | 34 | | 2.3 |
| MCT) | | | | |
| MUFA | g | 17 | | 1.1 |
| PUFA | g | 20 | | 1.3 |
| linoleic acid | g | 14 | | 0.92 |
| (n-6) | | | | |
| α linolenic | g | 2.0 | | 0.13 |
| acid (n-3) | | | | |
| Ratio ω6/ω3 | | 2.5 | | 2.5 |
| EPA | g | 2.1 | 1-5 g/day, | 0.14 |
| | | | or 1-7% wt | |
| | | | total | |
| | | | lipids | |
| DHA | g | 1.5 | | 0.10 |
| Mineral and | | | | |
| Trace | | | | |
| Elements | | | | |
| Sodium | mg | 2295 | | 153.0 |
| Potassium) | mg | 2700 | | 180.0 |
| Calcium | mg | 1140 | | 76.0 |
| Phosphorus | mg | 1275 | | 85.0 |
| Magnesium | mg | 405 | At least 15, | 27.0 |
| | | | preferably 15 | |
| | | | to 35 | |
| Chloride | mg | 2070 | | 138.0 |
| Iron | mg | 14 | | 0.93 |
| Zinc | mg | 28 | 0.4 to 2.0 | 1.9 |
| Copper | mg | 2.4 | | 0.16 |
| Fluoride | mg | 1.95 | | 0.13 |
| Chromium | μg | 115.5 | | 7.7 |
| Molybdenum | μg | 285 | | 19 |
| Selenium | μg | 135 | | 9 |
| Manganese | mg | 5.4 | | 0.36 |
| Iodine) | μg | 300 | | 20 |
| Vitamins | | | | |
| Vitamin A | IU | 8250 | | 550 |
| total | | | | |
| Vitamin D | μg | 30 | | 2.0 |
| Vitamin E | IU | 97.5 | | 6.5 |
| Vitamin K | μg | 180 | | 12 |
| Vitamin C | mg | 435 | | 29 |
| Vitamin B1 | mg | 4.05 | | 0.27 |
| (Thiamin) | | | | |
| Vitamin B2 | mg | 4.35 | | 0.29 |
| (Riboflavin) | | | | |
| Vitamin B3- | mg | 40.5 | 0.7 to 3.5 | 2.7 |
| PP | | | | |
| (Niacin) | | | | |
| Vitamin B5 | mg | 16.5 | 0.2 to 2.0 | 1.1 |
| (Pantothenic | | | | |
| acid) | | | | |
| Vitamin B6 | mg | 5.25 | 0.1 to 0.7 | 0.35 |
| (Pyridoxine) | | | | |
| Vitamin B8 | μg | 107 | At least 1 | 7.1 |
| (Biotin) | | | | |
| Vitamin B9 | μg | 840 | At least 12 | 56 |
| (Folic Acid) | | | | |
| Vitamin B12 | μg | 13.8 | 0.1 to 1 | 0.92 |

Example Nos. 3 and 4 below are designed to provide maintenance before diagnosis of a tumor (if needed) and after the tumor has gone into remission.

### Example No. 3

| | | Embodiment | Embodiment |
|---|---|---|---|
| | | Maintenance per | per |
| | | 1500 ml | per 100 ml |
| Calories | Kcal | 1875 | 125 |
| Protein | g | 62 | 4.1 |
| Ca Caseinate | g | 31 | 2.06 |
| Soya | g | 31 | 2.06 |
| Carbohydrates | g | 252 | 16.8 |
| Maltodextrins | g | 237 | 15.8 |
| Carbohydrates | g | 15 | 1.0 |
| from other sources | | | |
| Fiber | g | 23 | 1.52 |
| Mix 1 | | | |
| Insoluble | % | 66 | 66 |
| Soluble | % | 34 | 34 |
| Lipids | g | 72 | 4.8 |
| SFA | g | 11 | 0.73 |
| MUFA | g | 43 | 2.9 |
| PUFA | g | 11 | 0.73 |
| linoleic acid (n-6) | g | 8.4 | 0.56 |
| α linolenic acid (n- | g | 1.6 | 0.11 |
| 3) | | | |
| Ratio ω6/ω3 | | 5.2 | 5.2 |
| Minerals and | | | |
| Trace Elements | | | |
| Sodium | mg | 2400 | 160 |
| Potassium | mg | 2445 | 163 |
| Calcium | mg | 1290 | 86 |
| Phosphorous | mg | 855 | 57 |
| Magnesium | mg | 405 | 27 |
| Chloride | mg | 3225 | 215 |
| Iron | mg | 18 | 1.2 |
| Zinc | mg | 12 | 0.78 |
| Copper | mg | 2 | 0.13 |
| Fluoride | mg | 1.4 | 0.09 |
| Chromium | μg | 105 | 7.0 |
| Molybdenum | μg | 98 | 6.5 |
| Selenium | μg | 81 | 5.4 |
| Manganese | mg | 4.4 | 0.29 |
| Iodine | μg | 165 | 11 |
| Vitamins | | | |
| Vitamin A total | IU | 4500 | 300 |
| Vitamin D | μg | 20 | 1.3 |
| Vitamin E | IU | 48 | 3.2 |
| Vitamin K | μg | 105 | 7.0 |
| Vitamin C | mg | 180 | 12.0 |
| Vitamin B1 | mg | 2.0 | 0.13 |
| (Thiamin) | | | |
| Vitamin B2 | mg | 1.7 | 0.11 |
| (Riboflavin) | | | |
| Vitamin B3-PP | mg | 23 | 1.50 |
| (Niacin) | | | |
| Vitamin B5 | mg | 9.5 | 0.63 |
| (Pantothenic acid) | | | |
| Vitamin B6 | mg | 2.3 | 0.15 |
| (Pyridoxine) | | | |
| Vitamin B8 | μg | 57 | 3.8 |
| (Biotin) | | | |
| Vitamin B9 | μg | 450 | 30 |
| (Folic Acid) | | | |
| Vitamin B12 | μg | 5.7 | 0.38 |
| Other | | | |
| Choline | mg | 810 | 54 |
| Taurine | mg | 81 | 5.4 |
| Carnitine | Mg | 150 | 10 |
| Beta-carotene | mg | 3.8 | 0.25 |
| (carrot) | | | |
| Lycopene (tomato) | mg | 5.9 | 0.39 |

### Example No. 4

| | | Embodiment | | |
|---|---|---|---|---|
| | | Maintenance | RANGE | Embodiment |
| | | per 1500 ml | for 100 kcal | per 100 ml |
| Calories | kcal | 1875 | 0.8-1.4 kcal/ml | 125 |
| Protein | g | 62 | 10-18% of total | 4.1 |
| | | | energy content, | |
| | | | intact | |
| | | | or partially | |
| | | | hydolysed | |
| Ca Caseinate | g | 31 | | 2.06 |
| Soya | g | 31 | | 2.06 |
| Carbohydrates | g | 252 | 40-65% of total | 16.8 |
| | | | energy content | |
| Maltodextrins | g | 237 | | 15.8 |
| Carbohydrates | g | 15 | | 1.0 |
| from other | | | | |
| sources | | | | |
| Fibers | g | 23 | >10 g/litre | 1.5 |
| Insoluble | % | 66 | | 66 |
| Soluble | % | 34 | | 34 |
| Lipids | g | 72 | 25-40% of total | 4.8 |
| | | | energy content | |
| SFA | g | 11 | saturated fats (not | 0.73 |
| | | | inc. MCT) <10% of | |
| | | | total energy content; | |
| | | | or <1.11 g/100 kcal | |
| MUFA | g | 43 | | 2.9 |
| PUFA | g | 11 | | 0.73 |
| Linoleic acid | g | 8.4 | 3-10% of total | 0.56 |
| (n-6) | | | energy content | |
| | | | linoleic acid | |
| | | | or higher w6 | |
| | | | derivatives or | |
| | | | 0.33-1.11 g/100 kcal | |
| α linolenic | g | 1.6 | >0.6% of total | 0.11 |
| acid (n-3) | | | energy content or | |
| | | | >0.06 g/100 kcal | |
| Ratio ω6/ω3 | | 5.2 | 2-7 | 5.2 |
| Minerals and | | | | |
| Trace | | | | |
| Elements | | | | |
| Sodium | mg | 2400 | 100-200 | 160 |
| Potassium | mg | 2445 | 25-250 | 163 |
| Calcium | mg | 1290 | At least | 86 |
| | | | 50 preferably | |
| | | | 50-300 g | |
| Phosphorus | mg | 855 | <150 g preferably | 57 |
| | | | 40-80 | |
| Magnesium | mg | 405 | At least | 27 |
| | | | 15 preferably | |
| | | | 15-35 | |
| Chlorides | mg | 3225 | At least 100 g | 215 |
| | | | preferably 150-250 | |
| Iron | mg | 18 | 0.4-1.5 | 1.2 |
| Zinc | mg | 12 | 0.4-2.0 | 0.78 |
| Copper | mg | 2 | 0.08-0.4 | 0.13 |
| Fluoride | mg | 1.4 | <0.15 | 0.09 |
| Chromium | μg | 105 | 2-10 | 7.0 |
| Molybdenum | μg | 98 | 2-14 | 6.5 |
| Selenium | μg | 81 | 3-9 | 5.4 |
| Manganese | μg | 4.4 | 0.1-0.4 | 0.29 |
| Iodine | μg | 165 | 7-15 | 11 |
| Vitamins | | | | |
| Vitamin A | IU | 4500 | 100-500 inc. b- | 300 |
| Total | | | carotene | |
| Vitamin D | μg | 20 | 0.5-2.5 | 1.3 |
| Vitamin E | IU | 48 | 2.2-6 | 3.2 |
| Vitamin K | μg | 105 | Greater than 4 | 7.0 |
| | | | preferably 6-15 | |
| Vitamin C | mg | 180 | Greater than 4 | 12.0 |
| Vitamin B1 | mg | 2.0 | Greater than 0.06 | 0.13 |
| (Thiamin) | | | preferably 0.06-0.4 | |
| Vitamin B2 | mg | 1.7 | Greater than 0.07 | 0.11 |
| (Riboflavin) | | | | |
| Vitamin B3-PP | mg | 23 | 0.7-3.5 | 1.5 |
| (Niacin) | | | | |
| Vitamin B5 | mg | 9.5 | 0.2-2.0 | 0.63 |
| (Panthothenic | | | | |
| acid) | | | | |
| Vitamin B6 | mg | 2.3 | 0.1-0.7 | 0.15 |
| (Pyridoxine) | | | | |
| Vitamin B8 | μg | 57 | At least 1 | 3.8 |
| (Biotin) | | | | |
| Vitamin B9 | μg | 450 | At least 12 | 30 |
| (Folic Acid) | | | | |
| Vitamin B12 | μg | 5.7 | 0.1-1 | 0.38 |
| Other | | | | |
| Choline | mg | 810 | If present, >30 | 54 |
| Taurine | mg | 81 | If present >4 | 5.4 |
| Carnitine | mg | 150 | If present >3 | 10 |
| Beta-carotene | mg | 3.8 | >0.1 | 0.25 |
| (carrot) | | | | |
| Lycopene | mg | 5.9 | >0.2 | 0.39 |
| (tomato) | | | | |

By way of example and not limitation, in an embodiment of the present invention, one of the formulas set forth in Examples 1 and 2 will be administered to the patient in the period between diagnosis and re-establishment of normo-metabolic status. This product will be administered at least once a day. If desired, the glutamine can be provided as a separate module. Prior to diagnosis, if required, a patient can receive a maintenance formula set forth in Examples 3 and 4. Likewise, after the treatment is finished and the tumor is in remission the patient will receive the maintenance formula set forth in Examples 3 and 4.

In another embodiment of the method, after a diagnosis of cancer, 1500 ml per day of one of the formulas of Examples 1 and 2 will be administered to a patient. At least one week prior to a cancer treatment, a glutamine module with 30 g of glutamine will also be administered to the patient along with the formula. Two weeks after the completion of the treatments, the glutamine supplementation will stop and the formula administration will continue as needed.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for providing long term nutrition to a cancer patient comprising the steps of:
administering at least once a day long term through a tube to a cancer patient a nutritional product comprising:
a source of protein which provides 14 to 25% by caloric content of the product wherein the protein source comprises at least 50% by caloric content whey;
at least 0.5% by caloric content glutamine;
at least 0.8% by caloric content leucine;
a source of carbohydrates that comprises 20 to 55% by caloric content of the product; and
a source of lipids that comprises 25 to 40% by caloric content of the product.

2. The method of claim 1 wherein glutamine comprises 0.5 to 10.0% by caloric content of the product.

3. The method of claim 1 wherein at least a part of the glutamine is packaged in a separate container from remaining components of the product.

4. The method of claim 1 wherein the protein source is intact or partially hydrolyzed protein.

5. The method of claim 1 wherein the glutamine is bound to a protein.

6. The method of claim 1 wherein the glutamine is free as an amino acid.

7. The method of claim 1 wherein the glutamine is a dipeptide.

8. The method of claim 1 wherein the protein source is 100% whey protein.

9. The method of claim 1 wherein 3 to 7% by caloric content of the product is provided by glutamine.

10. The method of claim 1 wherein 1.5 to 3.5% by caloric content of the product is provided by leucine.

11. The method of claim 1 wherein the lipid source provides 30 to 35% by caloric content of the product.

12. The method of claim 1 wherein the energy density of the product is between 1.0 to 2.0 kcal.

13. The method of claim 1 wherein the product comprises a source of fiber.

14. The method of claim 1 wherein the patient receives a second different tube-fed nutritional product after normo-metabolic status has been re-established.

15. The method of claim 1 wherein the glutamine comprises alanyl-glutamine.

16. A method of providing long term tube-fed nutrition to a cancer patient comprising the steps of:
administering through a tube to a cancer patient with a tumour requiring treatment a first composition comprising: a source of protein which provides 14 to 25% by caloric content of the product wherein the protein source comprises at least 50% by caloric content whey, at least 0.5% by caloric content glutamine, at least 0.8% by caloric content leucine, a source of carbohydrates that comprises 20 to 55% by caloric content of the product, and a source of lipids that comprises 25 to 40% by caloric content of the product; and
administering through a tube to the cancer patient after the normo-metabolic status has been re-established a second long-term nutritional composition that has a different composition than the first composition.

17. The method of claim 16 wherein the second long-term nutritional composition comprises per 100 kcal of product:
a source of protein;
a source of carbohydrates;
a source of lipids;
sodium 100 to 200 mg;
potassium 25 to 250 mg;
calcium above 50 mg;
phosphorus less than 150 mg;
magnesium at least 15 mg;
chloride at least 100 mg;
iron 0.4 to 1.5 mg;
zinc 0.4 to 2.0 mg;
copper 0.08 to 0.4 mg;
fluoride 0 to 0.15 mg;
chromium 2.0 to 10.0 micrograms;
molybdenum 2.0 to 14.0 micrograms;
selenium 3.0 to 9.0 micrograms;
manganese 0.1 to 0.4 mg;
iodine 7.0 to 15.0 micrograms
Vit A 100 to 500 IU;
Vit D 0.5 to 2.5 micrograms;
Vit E 1.5 to 4.0 mg;
Vit K greater than 4.0 micrograms;
Vit C greater than 4.0 mg;
Vit B1 greater than 0.06 mg;
Vit B2 greater than 0.07 mg;
Vit B3 0.7 to 3.5 mg;
Vit B5 0.2 to 2.0 mg;
Vit B6 0.1 to 0.7 mg;
Vit B8 at least 1.0 micrograms;
Vit B9 at least 12.0 micrograms; and
Vit B12 0.1 to 1.0 micrograms.

18. The method of claim 16 wherein glutamine comprises 0.5 to 10.0% by caloric content of the product.

19. The method of claim 16 wherein glutamine is packaged in a separate container from remaining components of the product.

20. The method of claim 16 wherein the protein source is intact or partially hydrolyzed protein.

21. The method of claim 16 wherein the protein source is 100% whey protein.

22. The method of claim 16 wherein 3 to 7% by caloric content of the product is provided by glutamine.

23. The method of claim 16 wherein 1.5 to 3.5% by caloric content of the product is provided by leucine.

24. The method of claim 16 wherein the energy density of the product is between 1.0 to 2.0 kcal.

25. The method of claim 16 comprising a source of fiber.

26. A method of providing long-term nutrition through a tube to a cancer patient comprising the steps of
administering at least once a day long term of the diagnosis a nutritional composition comprising a source of protein which provides 14 to 25% by caloric content of the product wherein the protein source comprises at least 50% by caloric content whey; a source of carbohydrates that comprises 20 to 55% by caloric content of the product; and a source of lipids that comprises 25 to 40% by caloric content of the product; and
administering, at least one week prior to a cancer treatment, glutamine with the nutritional composition.

27. The method of claim 26 including the step of administering to the patient the glutamine as a separate module.

28. A composition for providing long term nutrition through a tube to a cancer patient comprising:
a source of protein which provides 14 to 25% by caloric content of the product wherein the protein source comprises at least 50% by caloric content whey;
at least 0.5% by caloric content glutamine;
at least 0.8% by caloric content leucine;
a source of carbohydrates that comprises 20 to 55% by caloric content of the product; and
a source of lipids that comprises 25 to 40% by caloric content of the product.

29. The composition of claim 28 wherein glutamine comprises 0.5 to 10.0% by caloric content of the product.

30. The composition of claim 28 wherein glutamine is packaged in a separate container from remaining components of the product.

31. The composition of claim 28 wherein 14 to 25% by caloric content of the protein source is intact or partially hydrolyzed protein.

32. The composition of claim 28 wherein the protein source is 100% whey protein.

33. The composition of claim 28 wherein 3 to 7% by caloric content of the product is provided by glutamine.

34. The composition of claim 28 wherein 1.5 to 3.5% by caloric content of the product is provided by leucine.

35. The composition of claim 28 wherein the lipid source provides 30 to 35% by caloric content of the product.

36. The composition of claim 28 wherein the energy density of the product is between 1.0 to 2.0 kcal.

37. The composition of claim 28 wherein the product comprises a source of fiber.
